(19) United States
(12) Patent Application Publication
(10) Pub. No.: US 2026/0202223 A1
(43) Pub. Date: Jul. 16, 2026

(54) GARMENT SENSOR SYSTEMS

(71) Applicant: Organic Robotics Corporation, Ithaca, NY (US)

(72) Inventors: Riley Edmonds, Ithaca, NY (US); Robert Shepherd, Ithaca, NY (US); Melanie Lyons, Maine, NY (US); Andres Serrano, Ithaca, NY (US); Ilayda Samilgil, Somerville, MA (US); Charles Cameron Abnet, Waltham, MA (US)

(21) Appl. No.: 19/135,783

(22) PCT Filed: Dec. 5, 2023
(86) PCT No.: PCT/US2023/082565

§ 371 Date: Jun. 4, 2025

Related U.S. Application Data
(60) Provisional application No. 63/430,236, filed on Dec. 5, 2022.

(51) Int. Cl. G01D 5/353; A61B 5/00

(52) U.S. Cl. CPC G01D 5/353; A61B 5/6804

(57) ABSTRACT

A sensor system includes a material having a stretchable region and a Stretch direction non-stretchable region and an optical fiber attached to the material. Fiber sections of the optical fiber are bonded together at a bond site on the optical fiber. The optical fiber is attached to the material such that the bond site is located in the non-stretchable region of the material. In some embodiments, the sensor system includes a material capable of stretching along a stretch direction and an optical fiber attached to the material. The optical fiber includes linear sections along its length. The linear sections are each angled relative to the stretch direction. When the material is stretched along the stretch direction, the angles of the linear sections relative to the stretch direction decrease and optical attenuation of the optical fiber decreases.

### CROSS-REFERENCE TO RELATED APPLICATION

This Patent Application claims priority to U.S. Provisional Patent Application No. 63/430,236, filed on Dec. 5, 2022, and entitled “Garment Sensor System.” The disclosure of the prior Application is considered part of and is incorporated by reference into this Patent Application.

### GOVERNMENT RIGHTS

This invention was made with Government support under FAIN 2139404 awarded by National Science Foundation. The government has certain rights in this invention.

### FIELD OF THE DISCLOSURE

This disclosure is directed to optoelectronic fiber sensor systems and methods.

### SUMMARY

The following description presents a simplified summary in order to provide a basic understanding of some aspects described herein. This summary is not an extensive overview of the claimed subject matter. It is intended to neither identify key or critical elements of the claimed subject matter nor delineate the scope thereof.

Optoelectronic fibers may be used in combination with a material, to create a sensor system, such as a garment-sensor system. When using optoelectronic fibers, hereafter referred to as optical fibers, on dynamic materials such as wearable materials, a problem arises when the material is stretchable, as such stretching can reduce the lifetime of the sensor system. Notably, if the optical fibers have weak points, such as welds or other bonding sites, and are attached onto the material in a region of the material that is stretchable, then the weak point of the optical fiber is subject to deterioration, and ultimately breaking, reducing the lifetime of the sensor system. Accordingly, there is a need for improved sensor systems.

According to a first aspect, a sensor system is provided. As an example, a garment-sensor system is discussed hereafter, although the sensor system is not intended to be limited to garments or fabrics. The garment-sensor system includes a material having a stretchable region and a non-stretchable region. The garment-sensor system includes an optical fiber attached to the material. Fiber sections of the optical fiber are bonded together at a bond site on the optical fiber and the optical fiber is attached (e.g., stitched, bonded, adhered) to the material such that the bond site on the optical fiber is located in the non-stretchable region of the material.

In some embodiments, a gradient between the stretchable region and the non-stretchable region of the material increases from a strain of around 0%-10% within the non-stretchable region and to a strain of up to 100% in the stretchable region. As used herein, “strain” may be a measure of a change in a dimension of a material or a region of a material divided by the original dimension (e.g., a percentage change). In some embodiments, the gradient increases in gradations of about 1%-10%. In some embodiments, the gradient is based at least in part on stitch density. In some embodiments, the fiber sections of the optical fiber are further bonded together at additional bond sites on the optical fiber and the optical fiber is attached (e.g., stitched) to the material such that the additional bond sites on the optical fiber are located in the non-stretchable region of the material. In some embodiments, the optical fiber is configured to measure strain.

In some embodiments, the non-stretchable region is capable of a strain of 20% or less under normal operating conditions. Normal operating conditions may include an elastic region of the material or any portion of the material (e.g., non-stretchable and stretchable regions). In some embodiments, normal operating conditions include strains that do not result in plastic or permanent deformation of the material or any portion of the material. In some embodiments, the non-stretchable region is capable of a strain of 15% or less under normal operating conditions. In some embodiments, the non-stretchable region is capable of a strain of 10% or less under normal operating conditions. In some embodiments, the non-stretchable region is capable of a strain of 5% or less under normal operating conditions. In some embodiments, the non-stretchable region is capable of 15% of the strain of the stretchable region under normal operating conditions. In some embodiments, the non-stretchable region is capable of 10% of the strain of the stretchable region under normal operating conditions. In some embodiments, the non-stretchable region is capable of 7% of the strain of the stretchable region under normal operating conditions. In some embodiments, the non-stretchable region is capable of 5% of the strain of the stretchable region under normal operating conditions.

In some embodiments, the stretchable region is capable of a strain of 1000% or less under normal operating conditions. In some embodiments, the stretchable region is capable of a strain of 100% or less under normal operating conditions. In some embodiments, the stretchable region is capable of a strain of 10% or less under normal operating conditions. In some embodiments, the stretchable region is capable of 95% of the total strain of the stretchable and non-stretchable regions under normal operating conditions. In some embodiments, the stretchable region is capable of 90% of the total strain of the stretchable and non-stretchable regions under normal operating conditions. In some embodiments, the stretchable region is capable of 80% of the total strain of the stretchable and non-stretchable regions under normal operating conditions.

In some embodiments, the non-stretchable region comprises a dense stitching pattern. In some embodiments, the dense stitching pattern comprises thread spacing of no more than 2 mm. In some embodiments, the dense stitching pattern comprises thread spacing of no more than 1 mm. In some embodiments, the dense stitching pattern comprises thread spacing of no more than 0.5 mm.

In some embodiments, the thread spacing of the dense stitching pattern is uniform. In some embodiments, the thread spacing of the dense stitching pattern is non-uniform. In some embodiments, the dense stitching pattern comprises an average thread spacing of no more than 2 mm. In some embodiments, the dense stitching pattern comprises an average thread spacing of no more than 1 mm. In some embodiments, the dense stitching pattern comprises an average thread spacing of no more than 0.5 mm.

In some embodiments, a first length of the optical fiber in a non-stretchable region on a first side of the weld site is at least 15 mm. In some embodiments, a second length of the optical fiber in the non-stretchable region on a second side of the weld site is at least 15 mm. In some embodiments, the optical fiber is substantially straight along the first length. In some embodiments, the optical fiber is substantially straight along the second length.

In some embodiments, a direction of the optical fiber along the first length is substantially perpendicular to a stretch direction of the material. In some embodiments, a direction of the optical fiber along the first length is off-parallel to a stretch direction of the material. In some embodiments, an angle between the direction of the optical fiber along the first length and the stretch direction of the material is less than 45°. In some embodiments, an angle between the direction of the optical fiber along the first length and the stretch direction of the material is less than 20°.

According to another aspect, a sensor system is provided having an optical fiber attached to a material. The material may stretch along a stretch direction and the optical fiber moves with the material as the material is stretched. The optical fiber includes linear sections along its length. The linear sections are each angled relative to the stretch direction. When the material is stretched along the stretch direction, the angles of the linear sections relative to the stretch direction decrease. As the angles become more obtuse, the measurable optical signal increases.

In some embodiments, the linear sections are separated by bends having bend angles. When the material is stretched along the stretch direction, the bend angles increase and the optical attenuation associated with each of the bends decreases. In some embodiments, the optical fiber comprises a first set of linear sections and a second set of linear sections. The linear sections of each of the first and second set of linear sections are separated by alternating bends. In some embodiments, the first and second sets of linear sections form a loop. The first set of linear sections forms a first side of the loop and the second set of linear sections forms a second side of the loop. In some embodiments, the first set of linear sections and the second set of linear sections are separated by an inner facing bend. In some embodiments, the first set of linear sections and the second set of linear sections are connected via two linear sections separated by an outer facing bend. In some embodiments, each of the first and second sets of linear sections comprises at least four linear sections. In some embodiments, the linear sections are angled greater than 45° relative to the stretch direction when the material is unstretched. In some embodiments, the optical fiber comprises a first end configured to be coupled to a light emitter and a second end configured to be coupled to a light detector. In some embodiments, the optical fiber comprises at least three segments bonded together comprising a first end segment, a second end segment, and one or more middle segments. In some embodiments, one or more middle segments comprises the linear sections. In some embodiments, the one or more middle segments comprises a material having greater stretchability than material used in the first and second end segments. In some embodiments, the one or more middle segments comprises a material having less optical transmissibility than material used in the first and second end segments. In some embodiments, the linear sections comprise at least nine linear sections. In some embodiments, the material has a stretchable region and a non-stretchable region. The linear sections, or in some embodiments, the one or more middle segments, are coupled to the stretchable region. In embodiments having the one or more middle segments, the first and second end segments are coupled to the non-stretchable region. Bond sites between the one or more middle segments and each of the first and second end segments are located in the non-stretchable region.

According to another aspect, a sensor system is provided having an optical fiber attached to a stretchable material. The material may stretch along a stretch direction. The optical fiber comprises a plurality of alternating bends along its length. The alternating bends comprise bend angles. The alternating bends of the optical fiber move with the material as the material is stretched in the stretch direction and the bend angles are increased. As the angles become more obtuse, the measurable optical signal increases.

In some embodiments, the bend angles are less than 50° when the material is unstretched. In some embodiments, the bend angles are less than 40° when the material is unstretched. In some embodiments, the optical fiber comprises a linear section between each adjacent pair of alternating bends. In some embodiments, the optical fiber comprises a first set of alternating bends and a second set of alternating bends. In some embodiments, the first and second sets of alternating bends form a loop. The first set of alternating bends forms a first side of the loop and the second set of alternating bends forms a second side of the loop. In some embodiments, each of the first and second sets of alternating bends comprise at least 3 alternating bends. In some embodiments, the first set of the alternating bends and the second set of alternating bends are separated by an inner facing bend. In some embodiments, the first set of alternating bends and the second set of alternating bends are connected via two alternating bends separated by an outer facing bend.

### BRIEF DESCRIPTION OF THE DRAWINGS

The present disclosure, in accordance with one or more various embodiments, is described in detail with reference to the following figures. The drawings are provided for purposes of illustration only and merely depict typical or example embodiments. These drawings are provided to facilitate an understanding of the concepts disclosed herein and should not be considered limiting of the breadth, scope, or applicability of these concepts. It should be noted that for clarity and ease of illustration, these drawings are not necessarily made to scale.

FIG. 1 shows an exemplary garment-sensor system, in accordance with some embodiments of the present disclosure.

FIG. 2A shows an exemplary garment-sensor system, in accordance with some embodiments of the present disclosure.

FIG. 2B shows an exemplary garment-sensor system, in accordance with some embodiments of the present disclosure.

FIG. 3 shows an exemplary garment-sensor system, in accordance with some embodiments of the present disclosure.

FIG. 4 shows an exemplary garment-sensor system, in accordance with some embodiments of the present disclosure.

FIGS. 5A and 5B show an at-rest and a stretched portion of an exemplary garment-sensor system, in accordance with some embodiments of the present disclosure.

FIG. 6A shows a mold for shaping an optical fiber, in accordance with some embodiments of the present disclosure.

FIG. 6B shows a mold for encapsulating an optical fiber, in accordance with some embodiments of the present disclosure.

FIGS. 7A-7D show schematic diagrams for forming an exemplary garment-sensor system, in accordance with some embodiments of the present disclosure.

FIG. 8 is a flowchart of an illustrative process for forming an exemplary garment-sensor system, in accordance with some embodiments of the present disclosure.

FIGS. 9A-9B show a mold for shaping and encapsulating an optical fiber, in accordance with some embodiments of the present disclosure.

FIG. 10 is a flowchart of an illustrative process for forming an exemplary garment-sensor system, in accordance with some embodiments of the present disclosure.

FIG. 11 shows an exemplary garment-sensor system, in accordance with some embodiments of the present disclosure.

FIGS. 12A-12C show a fiber deforming device for shaping an optical fiber, in accordance with some embodiments of the present disclosure.

FIGS. 13A-13E show a fiber deforming structure for shaping an optical fiber, in accordance with some embodiments of the present disclosure.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

By incorporating the features of some of the disclosed embodiments, the lifespan of a sensor system (e.g., a garment-sensor system) may be significantly improved, even under extreme misuse conditions. Additionally, in accordance with some of the disclosed embodiments, the sensitivity of the sensor signal to stretching (e.g., signal strength or signal-to-noise ratio) is increased.

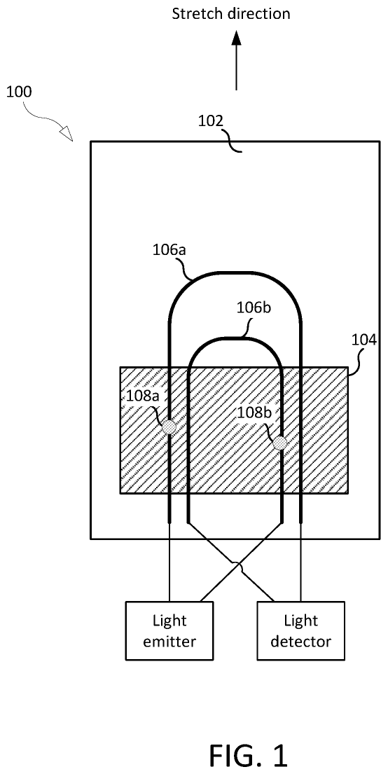

FIG. 1 shows an exemplary garment-sensor system 100 according to an embodiment. Garment-sensor system 100 includes a stretchable region 102 and a non-stretchable region 104. Optical fibers 106a, 106b are attached (e.g., stitched, bonded, adhered) to a material.

The material is capable of stretching along at least one stretch direction. In the embodiment depicted in FIG. 1, the material comprises a textile and is part of a garment or item of clothing and the stretch direction is longitudinal (as shown on the page). In other embodiments, the material may comprise any material that accommodates the stretchable region 102 and a non-stretchable region 104. For example, the material may comprise an elastic material such as any one of rubbers, plastics, fabrics and textiles, leather, cardboard, foams, or elastomers, to name a few examples. In some embodiments, the material may comprise multiple materials or different materials for the stretchable region 102 and a non-stretchable region 104. Fiber sections of each of the optical fibers 106a, 106b are bonded together at one or more bond sites 108a, 108b on the optical fiber. For example, fiber sections of the optical fiber may be welded, glued, heat-shrink tubed, or otherwise fastened. Optical fibers 106a, 106b are attached (e.g., stitched, bonded, adhered) to the material such that the bond site on the optical fibers is located in the non-stretchable region 104. In some embodiments, the optical fibers 106a, 106b are attached to the material using a polymer casing. In some embodiments, a polymer casing is used to bond fiber sections of the optical fibers 106a, 106b together at the bond sites 108a, 108b. In some embodiments, the polymer casing comprises rubber.

Stretchable region 102 may be highly stretchable (e.g., hyperelastic), and be capable of a strain up to 500% under normal operating conditions. In some embodiments, stretchable region 102 may be capable of a strain over 20% under normal operating conditions, and in some embodiments, may be capable of strains between 20% and 500% under normal operating conditions, such as between 100% and 500%, and in general is more stretchable than the non-stretchable region 104. In some embodiments, non-stretchable region 104 may be inextensible, or capable of a strain from about 0% to about 20% under normal operating conditions, or in some embodiments from about 0% to about 15%, or from about 0% to about 10%, or from about 0% to about 5%. Each of the stretchable region 102 and non-stretchable region 104 may have uniform elasticity, or non-uniform elasticity. For example, there may be a gradient that increases, for example, from about 0% to 10% in the non-stretchable region 104 and increases to about 100% in the stretchable region 102. As a further example, the gradient may increase in gradations of about 1%-10%.

As shown in FIG. 1, there is a single stretchable region 102 and a single non-stretchable region 104. This is for illustrative purposes, and in some embodiments, there may be more regions than this example, and the regions may have different shapes than as shown in FIG. 1.

Optical fibers 106a, 106b may be capable of detecting strain. There may be fewer than two optical fibers 106a, 106b, or there may be more, and the path of the optical fibers may be different than as shown in FIG. 1. In some embodiments, optical fibers 106a, 106b may be the optical fibers disclosed in WO 2022/216736, a co-owned PCT application, the contents of which are incorporated herein in their entirety. The optical fibers 106a, 106b comprise a first end configured to be coupled to a light emitter and a second end configured to be coupled to a light detector.

In some embodiments, optical fibers 106a, 106b may comprise an acrylic portion or segment having a high optical-index-of-refraction core, a low optical-index-of-refraction cladding, and a jacketing material that forms a protective jacket meant to protect the fiber's cladding and core. For example, the jacket may be a plastic, such as polyethylene. The optical fibers 106a, 106b may also comprise a rubber portion or segment, such as where the optical fibers 106a, 106b have a significant curvature. The rubber portion, for example, may have a core that comprises polyester and/or polyurethane, a silicone cladding, and a silicone jacket. Each portion (e.g., acrylic or rubber) may have several sections. For example, the rubber portion may be shaped to include a plurality of linear sections oriented in different directions or orientations. In the embodiment depicted in FIG. 1, the rubber portion of optical fiber 106a, which is located above the bond site 108a (as shown on the page), has two longitudinal sections (extending in the stretch direction) and a lateral section (orthogonal to the stretch direction). The materials used may be dependent on the intended use of the optical fibers and of the garment-sensor system, and the materials noted here are exemplary and non-limiting.

In one example of a manufacturing process, fiber sections of the optical fibers 106a, 106b are bonded (e.g., welded) at bond sites 108a, 108b and attached to the non-stretchable region 104. After being bonded (e.g., welded), the optical fibers 106a, 106b may be stitched or embroidered onto the textile of the material. In one embodiment, the non-stretchable region 104 is created prior to stitching the optical fibers 106a, 106b to the material. In some embodiments, it is also possible that an inextensible fabric could be connected to a stretchable fabric and the optical fibers 106a, 106b could be stitched across the different fabric panels.

In some embodiments, the bond may be a weld made by melting the polyester and/or polyurethane core of the rubber portion of the optical fibers 106a, 106b, and pushing a thermally softened (but not-molten) acrylic portion, such as the high optical-index-of-refraction core, into the thermally softened polyester/polyurethane core. After cooling, the surface of the acrylic may be heat bonded to the inside of the rubber core. In this way, in some embodiments, fiber sections of the optical fiber may be bonded together. In some embodiments, the longer the length of that bond, the more durable the connection of the optical fiber 106a, 106b to the material.

In the embodiment depicted in FIG. 1, the acrylic portion of the optical fibers 106a, 106b is coupled to the light emitter, the acrylic portion is bonded to the rubber portion, and the rubber portion is coupled to the light detector. In some embodiments, the size (e.g., cross-sectional area) of the core of the acrylic portion is different than the size of the core of the rubber section. In such embodiments, the core of the acrylic section may be smaller (e.g., have a smaller cross-sectional area) than the core of the rubber section. Light travels through the core of the acrylic portion and spreads out into the larger area core of the rubber portion. In some embodiments, the light may travel through a bond site 108a, 108b having a reduction in core size. In such embodiments, the light may be reflected back into the larger core size.

In some embodiments, the acrylic portion is bonded to the rubber portion, the rubber portion is bonded to a different acrylic portion, and the different acrylic portion is coupled to the light detector. In such embodiments, the core size may increase in each portion. In some embodiments, the core size of the acrylic portions may be smaller than the core size of the rubber section. In some embodiments, the rubber portion is coupled to the light emitter, the rubber portion is bonded to the acrylic portion, and the acrylic portion is coupled to the light detector.

In some embodiments, the non-stretchable region 104 of the material may be created by using a dense stitching pattern. For example, the thread spacing between threads may be no more than 2 mm in some embodiments, or no more than 1 mm in some embodiments. The density may be uniform or non-uniform. As discussed above, in some embodiments, there may be a gradient, and the gradient may in some embodiments be based on the density of the stitching. For example, in one area of the non-stretchable region 104, the thread spacing may be no more than 1 mm (e.g., closer to the weld site), while in another area (e.g., further away from the weld site), the thread spacing may increase to 2 mm.

In some embodiments, a high elastic modulus rubber is used to pattern the non-stretchable region 104. The high elastic modulus rubber may have an elastic modulus of at least 5 MPa. In such embodiments, a sensing region, such as the region of stretchable region 102 where the optical fibers 106a and 106b attach, may be encapsulated with the high elastic modulus rubber using a gradient porosity, which may allow stretching in the sensing region.

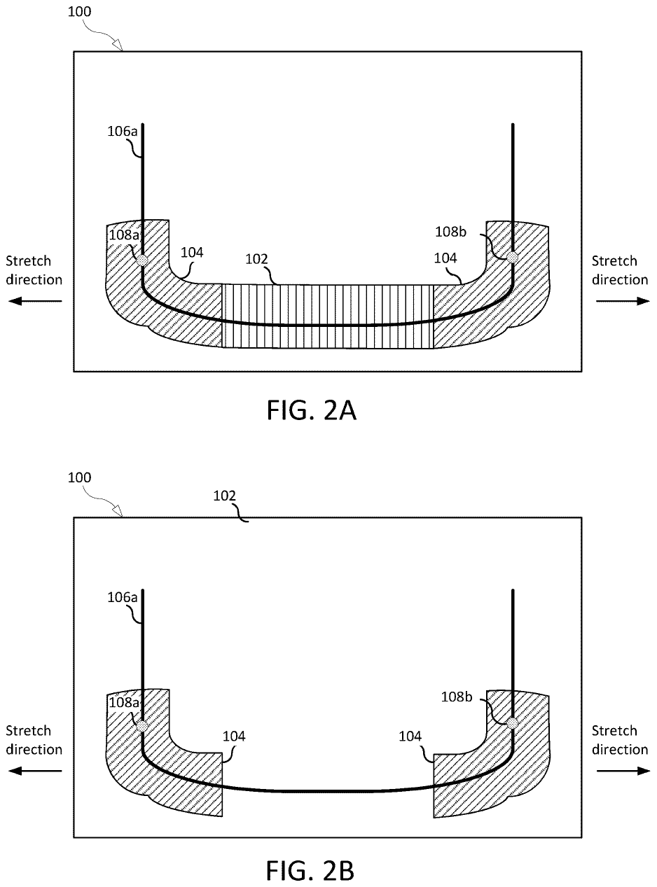

FIG. 2A shows an exemplary garment-sensor system 100 according to an embodiment. As shown in FIG. 2A, the shape of the non-stretchable region 104 is different than shown in FIG. 1A. In the embodiment depicted in FIG. 2A, there are two non-stretchable regions 104, separated by the stretchable region 102. Further, the dense stitching pattern of the non-stretchable region 104 transitions into the stretchable region 102 and varies over the stretchable region 102, which is located between the two non-stretchable regions 104 as indicated by the different cross-hatching in the figure. Closer to the bond sites 108a, 108b, the stitching pattern of the non-stretchable region 104 is relatively denser (e.g., about 1 mm between stitches), while in the stretchable region 102 located further away from the bond sites 108a, 108b the stitching pattern is not as dense (e.g., about 2 mm between stitches).

The rubber portion of the optical fiber 106a is attached to the two non-stretchable regions 104 and the stretchable region 102. In the embodiment depicted, the material is stretched in a stretch direction. As the material is stretched, the optical fiber 106a changes from a rounded or arced shape in the stretchable region 102 to a straight line. Thus, the garment-sensor system 100 depicted in FIG. 2A increases the optical attenuation and decreases the measured optical intensity as the optical fiber is stretched. In some embodiments, the stretchable region 102 constrains deformation or strain to a particular direction. For example, the stretchable region 102 may constrain stretching to the depicted stretch direction and restrict stretching in a different direction, such as a direction angled to or orthogonal to the depicted stretch direction.

In some embodiments, the stretchable region 102 in FIG. 2A is capable of a strain of 100% or less under normal operating conditions. In some embodiments, the stretchable region 102 is capable of a strain of 50% or less under normal operating conditions. In some embodiments, the stretchable region 102 is capable of a strain of 10% or less under normal operating conditions.

FIG. 2B shows an exemplary garment-sensor system 100 according to an embodiment. FIG. 2B is similar to the embodiment of FIG. 2A, except that the stretchable region 102i in the embodiment of FIG. 2B is different than the stretchable region 102 in FIG. 2A. For example, the stretchable region 102 does not include the stitching pattern. Accordingly, there are two non-stretchable regions 104, separated by an area of the stretchable region 102. In some embodiments, the stretchable region 102 in FIG. 2B may have similar strain properties or characteristics as the stretchable region 102 discussed in relation to FIG. 1. As the material is stretched in the stretch direction, the optical fiber 106a changes from a rounded or arced shape in the stretchable region 102 to a straight line, and the optical attenuation is increased and the measured optical intensity is decreased.

In some embodiments, there is a trade-off that must be made in designing a garment-sensor system as described herein. To properly function, the material (particularly when the optical fibers 106a, 106b are configured to measure strain) must be able to stretch. But too much stretch, particularly as applied to the bond sites 108a, 108b of the optical fibers 106a, 106b, will reduce the lifetime of the garment-sensor system. Accordingly, the application, expected duration of use, and number of cycles of strain, among other parameters, may be taken into account when selecting materials and considering the design of the stretchable and non-stretchable regions.

In some embodiments, it is desirable for there to be a minimum amount of the optical fiber 106a, 106b within the non-stretchable region 104 prior to the bond site. Similarly, in some embodiments, it is desirable for there to be a minimum amount of the optical fiber 106a, 106b within the non-stretchable region 104 after the bond site and before a significant curvature of the optical fiber 106a, 106b. This minimum amount helps to protect the bond site from excessive strain. For example, in some embodiments, there is at least 15 mm of jacketed fiber on the dense embroidered region before the bond site, followed by at least 15 mm of the rubber section after the bond site but before a curve. Additionally, in some embodiments, this approximately 30 mm segment is in straight line approximately perpendicular to the angle of stretch to reduce as much strain as possible being applied to the axis of the bonding region. In still other embodiments, this approximately 30 mm segment is in straight slightly off parallel to the angle of stretch to reduce as much strain as possible being applied to the axis of the bonding region.

In some embodiments, the bond sites 108a, 108b of the optical fibers 106a, 106b are positioned on the material such that the bond sites 108a, 108b are orthogonal to the direction of stretch of the material. In some embodiments, the bond sites 108a, 108b of the optical fibers 106a, 106b are positioned on the material such that the bond sites 108a, 108b are at an angle α with respect to the direction of stretch of the material, where one or more of the following conditions holds: (i) 0≤α≤90°, (ii) 0≤α≤45°, (iii) 0≤α≤15°, and (iv) 0≤α≤5°.

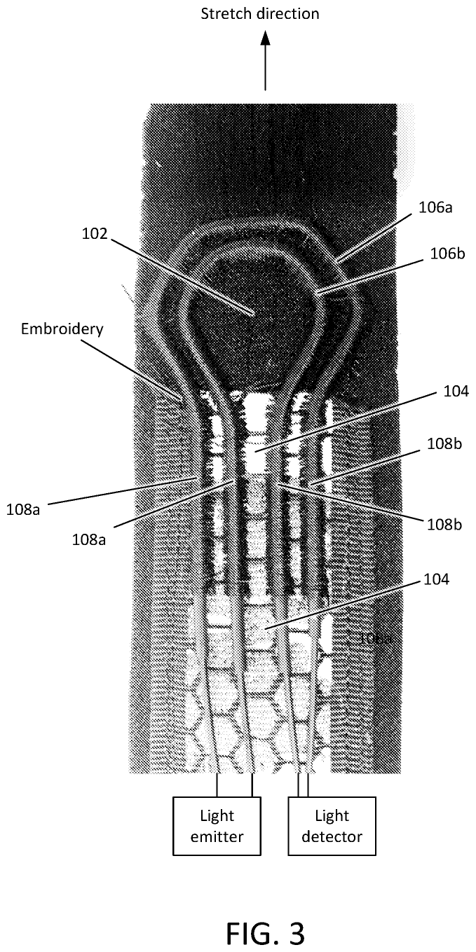

FIG. 3 shows an exemplary garment-sensor system, in accordance with some embodiments of the present disclosure. As shown in FIG. 3, the optical fibers 106a, 106b are bonded together at one or more bond sites 108a, 108b on the optical fiber. The bond sites 108a, 108b are located in the non-stretchable region 104. The optical fibers 106a, 106b are stitched or embroidered onto the stretchable region 102 and non-stretchable region 104 of the material. The optical fibers 106a, 106b comprise a first end configured to be coupled to a light emitter and a second end configured to be coupled to a light detector.

The rubber portion or segment of each optical fiber 106a, 106b may comprise linear sections along its length. The linear sections are each angled relative to the stretch direction and are separated by bends having bend angles. A linear section may be a may be section of the optical fiber 106a that is approximately straight. The linear section may include minor bends that have a minimal affect optical attenuation. The linear sections and bend angles form a different shape than a sinusoidal wave, which has alternating semi-circular curves. In some embodiments, the linear sections are at least the same length as the bends, such as at least twice the length of the bends, such as at least five times the length as the bends, such as at least ten times the length as the bends.

In the embodiment depicted in FIG. 3, optical fiber 106a has five linear sections that are oriented to form five sides of a hexagon-like shape. When the material is stretched along the stretch direction, the angles of the linear sections relative to the stretch direction change and the optical attenuations of the optical fiber 106a also change. In general, as a bend angle increases, the optical attenuation associated with the bend decreases. Optical fiber 106b has a generally curved shape in stretchable region 102. When the material is stretched along the stretch direction, the shape of optical fiber 106b changes and the optical attenuation of optical fiber 106b changes. In general, the changes in the shapes of optical fibers 106a, 106b when the material is stretched along the stretch direction may result in a decrease of the optical attenuation of the optical fibers 106a, 106b.

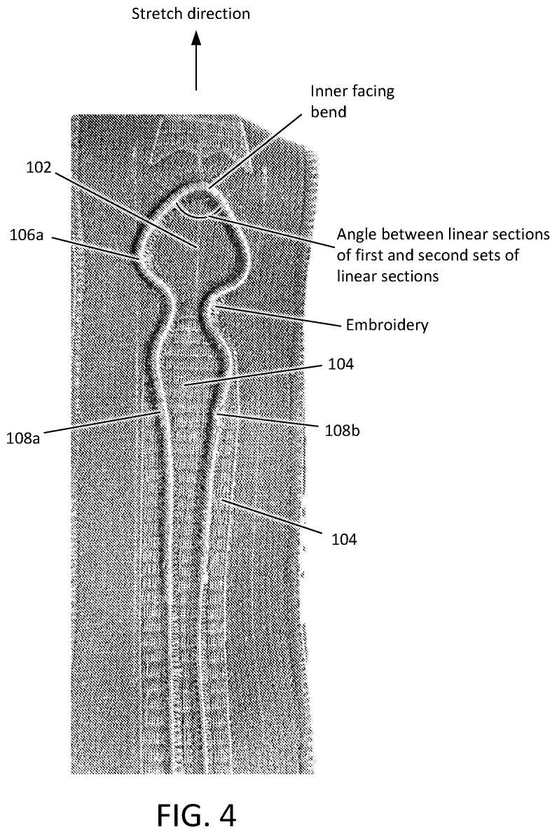

FIG. 4 shows an exemplary garment-sensor system, in accordance with some embodiments of the present disclosure. As shown in FIG. 4, there is only one optical fiber 106a. The rubber portion or segment of the optical fiber 106a has a number of linear sections that are different than what is shown in FIG. 3. The optical fiber 106a comprises at least three segments bonded together comprising a first end segment (e.g., an acrylic portion), a second end segment (e.g., an acrylic portion), and one or more middle segments (e.g., rubber portion(s)). The middle segment of the optical fiber 106a generally comprises four linear sections that are oriented to form a loop having a diamond-like shape in stretchable region 102. In some embodiments, the middle section comprises a material having greater stretchability than material used in the first and second end segments. In some embodiments, the middle segments comprise a material having less optical transmissibility than material used in the first and second end segments.

A first set of the linear sections (e.g., the leftmost two as shown on the page) form a first side of a loop and a second set of the linear sections (e.g., the rightmost two as shown on the page) form a second side of the loop. The first set of linear sections and the second set of linear sections are separated by an inner facing bend. For example, the first set of linear sections and the second set of linear sections are connected via two linear sections separated by the inner facing bend. The two linear sections have an angle that faces inward between them. When the material is stretched in the stretch direction, the angle between the linear segments of each of the first and second sides increases and the angle between the two linear sections separated by the inner facing bend may decrease. The overall change in angles may result in a decrease of the optical attenuation of the optical fiber 106a or an increase in the measurable optical signal.

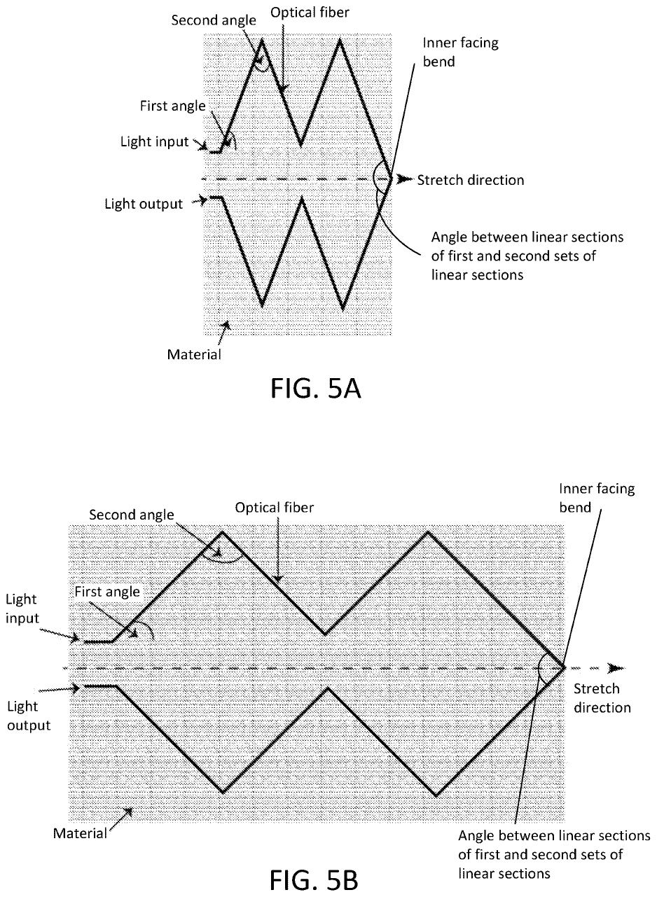

FIGS. 5A and 5B show an at-rest and a stretched portion of an exemplary garment-sensor system, in accordance with some embodiments of the present disclosure. In particular, FIG. 5A shows the at-rest portion and FIG. 5B shows the stretched portion, and are described together for clarity.

An optical fiber (e.g., optical fiber 106a, 106b in FIGS. 1-4) is attached to a material capable of stretching along a stretch direction. The material comprises a stretchable region (e.g., stretchable region 102 in FIGS. 1-4) and a non-stretchable region (e.g., non-stretchable region 104 in FIG. 104). The stretchable region of the material is shown in FIGS. 5A and 5B. Light may be input into a first end of the optical fiber (e.g., by a light emitter) and output at a second end of the optical fiber (e.g., to a light detector).

The optical fiber comprises a plurality of alternating bends along its length and a linear section between each adjacent pair of alternating bends. The alternating bends comprise bend angles, such as the labelled second angle. The linear sections form a loop in the optical fiber. A first set of linear sections (e.g., linear sections above the stretch direction arrow as shown on the page) form a first side of the loop and a second set of linear sections (e.g., linear sections below the stretch direction as shown on the page) form a second side of the loop. The linear sections of each of the first and second sets of linear sections are separated by the alternating bends. For example, the linear sections may form a “zig-zag” pattern.

The first set of linear sections and the second set of linear sections are connected via two linear sections separated by an inner facing bend (e.g., located on the stretch direction arrow). For example, the inner facing bend forms an angle between the two linear segments that is on an inside of the loop. Each of the first and second sets of linear sections comprises at least four linear sections (four sections are shown in the depicted embodiment). In some embodiments, the linear sections are positioned such that when the material is unstretched, the linear sections are angled at a first angle greater than 45° relative to the stretch direction. The bend angles of the alternating bends, shown as the second angle between the linear sections the first set of linear sections, may be at an angle that is twice the difference of 90° and the first angle. As the material is stretched in the stretch direction, the first angle (e.g., angle relative to the stretch direction) decreases, the second angle (e.g., bend angles) increases, and the optical attenuation of the optical fiber decreases. In some embodiments, the bend angles are 90° or less when material is unstretched, such as 60° or less, such as 45° or less. In some embodiments, the bend angles are 60° or more when the material is stretched under normal operating conditions, such as 90° or more, such as 120° or more. In some embodiments, the bend angles may be between 90° to 150° under normal operating conditions. In some embodiments, the bend angles may be between 45° to 70° under normal operating conditions. In some embodiments, under normal operating conditions, the bend angles may be between 60° to 120° under normal operating conditions.

In some embodiments, the greater the angle the optical fiber is relative to the stretching direction (e.g., the first angle), the greater the measurable optical signal (e.g., the light output) varies with strain. The greater the angle, the less optical signal is initially detectable as more optical rays exceed the critical angle of refraction. As the optical fiber becomes more aligned with the stretch direction, more optical rays are contained within the fiber and the measured optical signal is increased.

Incorporating an additional bend in the optical fiber that is non-parallel to the stretching direction increases the sensitivity to stretching. The more acute the angles of the bend, the more sensitive the optical signal is to stretching. Increasing the number of non-parallel angles (e.g., labelled first angle) of the optical fiber to the stretching direction may increase the sensitivity to stretching. In some embodiments, the optical fiber comprises at least five, seven, or nine non-parallel angles with the fiber stretching direction, where the majority of angles are <40° and cause a signal variation of 1,000 analog to digital input counts over a stretch of >5 cm.

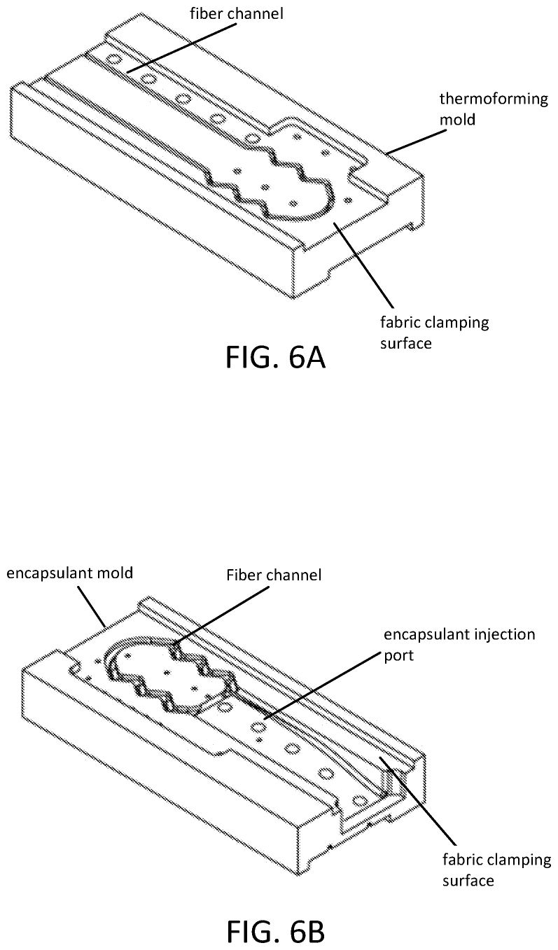

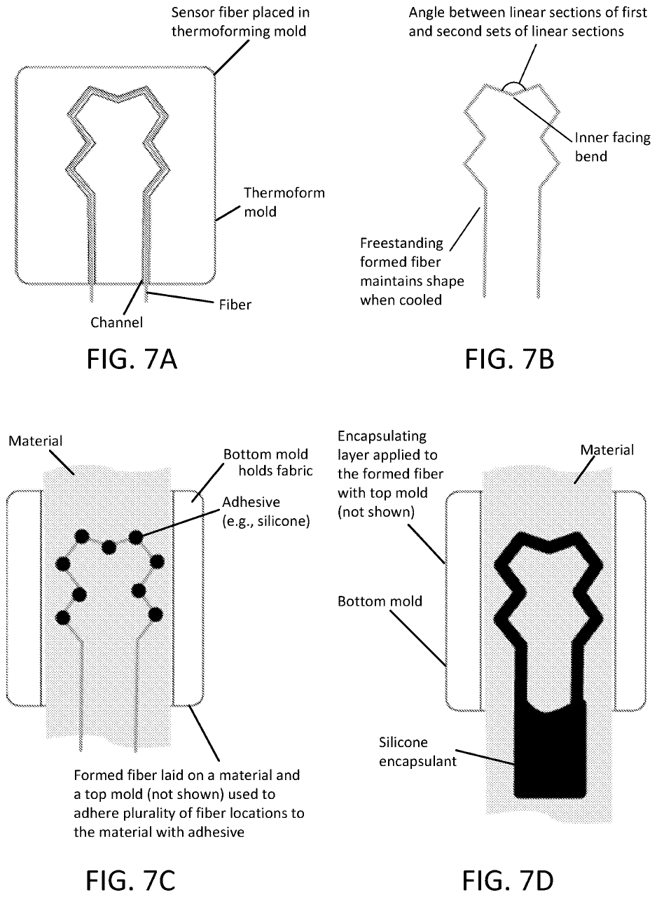

FIGS. 6A and 6B show mold blocks for forming a garment-sensor system, in accordance with some embodiments of the present disclosure. FIG. 6A shows a mold block for shaping an optical fiber (e.g., optical fiber 106a, 106b in FIGS. 1-4). The optical fiber is placed in a fiber channel of the mold and processed, such as discussed below in relation to the thermoforming mold of FIG. 7A, to form a freestanding optical fiber having the pattern or shape of the fiber channel. The optical fiber includes a first set of at least three alternating bends (five alternating bends are shown in the depicted embodiment) that form a first side of a loop and a second set of at least three alternating bends (five alternating bends are shown in the depicted embodiment) that form a second side of the loop. The first and second set of alternating bends are separated by an inner facing bend.

FIG. 6B shows a mold for encapsulating the optical fiber. The freestanding optical fiber may be placed in a fiber channel of the encapsulant mold and a material, such as a fabric, may be placed on a fabric clamping surface of the mold. Encapsulant is injected, such as discussed below in relation to the top mold of FIG. 7D, through encapsulant injection ports to encapsulate the optical fiber and adhere the optical fiber to the material.

FIGS. 7A-7D show schematic diagrams for forming an exemplary garment-sensor system, in accordance with some embodiments of the present disclosure. In the embodiment depicted in FIGS. 7A-7D, an optical fiber (e.g., optical fiber 106a, 106b in FIGS. 1-4) is shaped, attached to a material, and encapsulated.

Referring to FIG. 7A, a base sensor form can be produced by laying the optical fiber, such as an elastomer fiber, into a channel of a thermoform mold. The channel is used to form a desired sensor shape of the optical fiber. The thermoform mold material may comprise aluminum, steel, polytetrafluoroethylene, polyoxymethylene, or other appropriate materials compatible with the process. While the optical fiber is in the channel, sufficient heat is applied to soften the optical fiber. After application of heat, and when the optical fiber cools, the optical fiber form is maintained as a freestanding shape as shown in FIG. 7B.

The optical fiber of the exemplary garment-sensor system includes a first set of linear sections separated by alternating bends that form a first side of a loop and a second set of linear sections separated by alternating bends that form a second side of the loop. The first set of linear sections and the second set of linear sections are separated by an outer facing bend. For example, the first set of linear sections and the second set of linear sections are connected via two linear sections separated by the outer facing bend. The two linear sections have an angle that faces outward between them. For example, the angle is on an outside of the loop. When the optical fiber is stretched in a stretch direction, the angle between the linear segments of each of the first and second sides increases and the angle between the two linear sections separated by the outer facing bend may increase. The alternating bends in the optical fiber modulate the optical transmission intensity when the fiber shape is deformed (e.g., pulled in the stretch direction). For example, as the shape of the optical fiber is elongated, the bend angles will become more obtuse and the measured optical intensity will increase. The angle between the two linear facing sections may also become more obtuse and increase the measured optical intensity.

Referring to FIGS. 7C and 7D, a two-part mold (e.g., a bottom mold and a top mold) is used to position the formed optical fiber and a material, such as a fabric substrate. In this example, the fabric is an elastic strap. The bottom mold holds the fabric and the top mold (not shown) provides a pattern to receive liquid adhesive or elastomer. In the figure, the top mold is used to produce a plurality of adhesive dots to adhere the formed sensor (e.g., formed optical fiber) to the strap. In some embodiments, the formed optical fiber is placed on the fabric before the adhesive dots are applied such that the adhesive dots are placed over the optical fiber. In some embodiments, the adhesive dots are applied to the fabric and the optical fiber is placed on the adhesive dots before the adhesive cures such that the adhesive is located between the fabric and the optical fiber.

Referring to FIG. 7D, an additional mold pattern is used to apply an encapsulate to encapsulate the formed sensor. In some embodiments, the top mold comprises the additional mold pattern. In some embodiments, the top mold is similar to the encapsulant mold discussed in relation to FIG. 6B. In such embodiments, the fabric clamping surface of the encapsulant mold is placed on the material, which is held by the bottom mold, and the mold pattern (e.g., fiber channel of the encapsulant mold) is placed over the formed optical fiber.

The encapsulant is injected into the encapsulant injection port(s) of the encapsulant mold to encapsulate the formed sensor. The encapsulant protects the sensor and reduces noise from ambient light. The encapsulant also provides strain relief for the fiber ends extending to a control pod or control circuitry (not shown), or to and from a light emitter and detector. In some embodiments, the encapsulant mold forms the pattern to receive liquid adhesive or elastomer and produce the adhesive dots.

In some embodiments, the step of adhering the optical fiber to the fabric, as depicted in FIG. 7C, is not necessary. The encapsulant step, as depicted in FIG. 7C, may both adhere and encapsulate the optical fiber. In some embodiments, the adhesion step of FIG. 7C is used to maintain the shape of the optical wire such that the encapsulant viscosity does not displace the optical wire during molding.

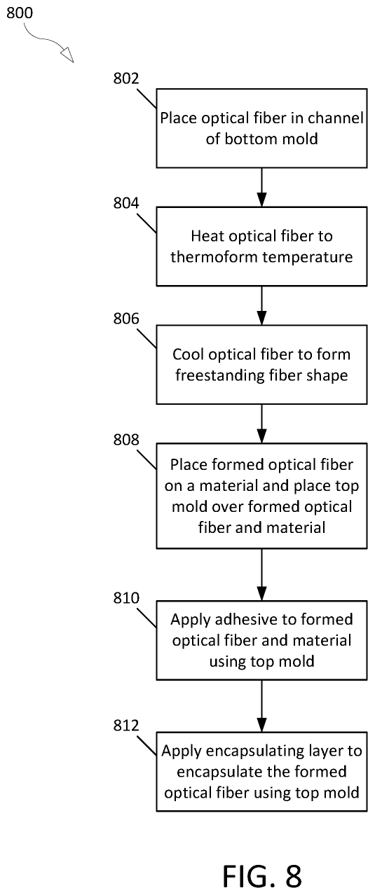

FIG. 8 is a flowchart of an illustrative process 800 for forming an exemplary garment-sensor system, in accordance with some embodiments of the present disclosure. The process 800 may be used to form the garment-sensor system discussed in relation to FIGS. 7A-7D.

The process 800 begins at operation 802 with placing an optical fiber (e.g., optical fiber 106a, 106b in FIGS. 1-4) in a channel of a bottom mold.

The process 800 continues to operation 804 with heating the optical fiber to a thermoform temperature. The thermoform temperature is the temperature or temperature range required to soften the optical fiber such that when the optical fiber is cooled, the optical fiber forms a freestanding shape when removed from the bottom mold. The thermoform temperature may vary depending on a material(s) of the optical fiber.

The process 800 continues to operation 806 with cooling the optical fiber to form a freestanding fiber shape. In some embodiments, the optical fiber may be cooled by surrounding air, such as by removing the heat and letting the optical fiber cool. In some embodiments, forced convection may be used to cool the optical fiber. In some embodiments, the bottom mold may comprise a cooling system that is used to cool the bottom mold and optical fiber.

The process 800 continues to operation 808 with placing the formed optical fiber on a material and placing a top mold (e.g., encapsulate mold in FIG. 6B) over the formed optical fiber and the material.

The process 800 continues to operation 810 with applying adhesive to the formed optical fiber and material using the top mold, such as discussed in relation to FIG. 7C.

The process 800 continues to operation 812 with applying an encapsulating layer to encapsulate the formed optical fiber using the top mold, such as discussed in relation to FIG. 7D.

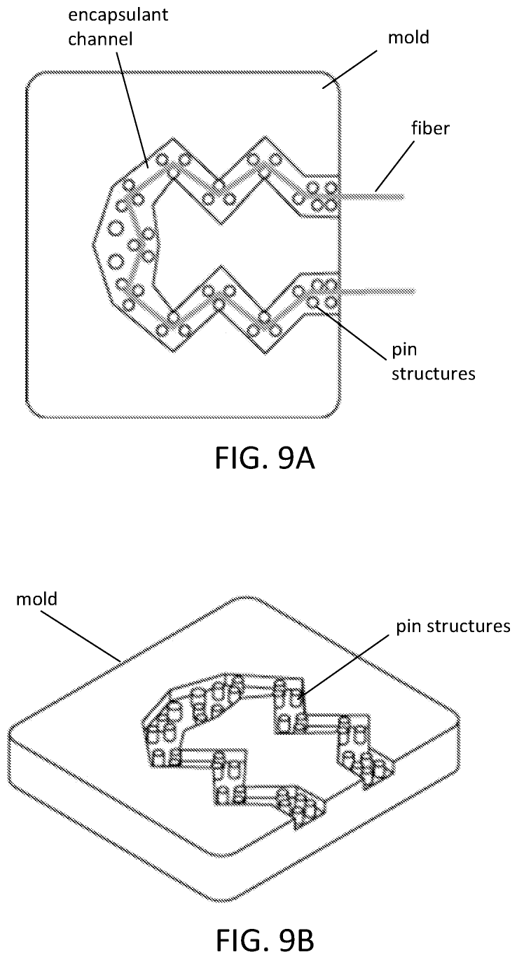

FIGS. 9A and 9B show a mold for shaping and encapsulating an optical fiber (e.g., optical fiber 106a, 106b in FIGS. 1-4), in accordance with some embodiments of the present disclosure. FIGS. 9A and 9B show different perspectives of the thermoforming mold and are described together for clarity. In the embodiment depicted in FIGS. 9A and 9B, an optical fiber is shaped, encapsulated, and attached to a material.

The mold forms a large channel and comprises thin pin structures disposed in the channel that are used to shape the optical fiber. For example, the optical fiber is routed through the pin structures to achieve the desired shape. The large channel has a volume that surrounds the pin structures and receives an encapsulant to encapsulate the optical fiber.

When the encapsulate is cured, the encapsulated optical fiber is ejected or lifted from the mold and placed on a material, such as a fabric. The cured encapsulant forms exposed volumes or recesses produced by the pins. The recesses may provide additional locations for adhesive to secure the encapsulated optical fiber to the fabric.

In some embodiments, the mold is a thermoforming mold. In such embodiments, the mold holds the optical fiber and heat is applied to soften the optical fiber. When cooled, the optical fiber forms a freestanding shape. The freestanding optical fiber may remain in the thermoforming mold to be encapsulated, or may be removed.

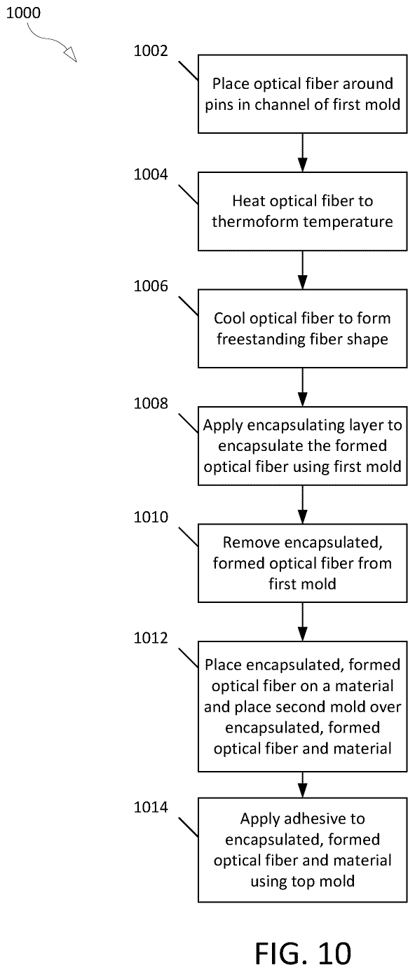

FIG. 10 is a flowchart of an illustrative process 1000 for forming an exemplary garment-sensor system, in accordance with some embodiments of the present disclosure. The process 1000 may be used to form a garment-sensor system using the thermoforming mold discussed in relation to FIGS. 9A and 9B.

The process 1000 begins at operation 1002 with placing an optical fiber (e.g., optical fiber 106a, 106b in FIGS. 1-4) around pins (e.g., pin structures in FIGS. 9A and 9B) in a channel (e.g., encapsulant channel in FIG. 9A) of a first mold, such as the thermoforming mold discussed in relation to FIG. 9A.

The process 1000 continues to operation 1004 with heating the optical fiber to a thermoform temperature. In some embodiments, the optical fiber may be heated as discussed in relation to operation 804 of FIG. 8.

The process 1000 continues to operation 1006 with cooling the optical fiber to form a freestanding fiber shape. In some embodiments, the optical fiber may be heated as discussed in relation to operation 806 of FIG. 8.

The process 1000 continues to operation 1008 with applying an encapsulating layer (e.g., silicone encapsulant in FIG. 7D) to encapsulate the formed optical fiber using the first mold.

The process 1000 continues to operation 1010 with removing the encapsulated, formed optical fiber from the first mold.

The process 1000 continues to operation 1012 with placing the encapsulated, formed optical fiber on a material and placing a second mold over the encapsulated, formed optical fiber and the material. In some embodiments, the second mold may be similar to the top mold discussed in relation to FIG. 7C.

The process 1000 continues to operation 1014 with applying an adhesive to the encapsulated, formed optical fiber and the material using the top mold. The adhesive may adhere the encapsulated, formed optical fiber to the material.

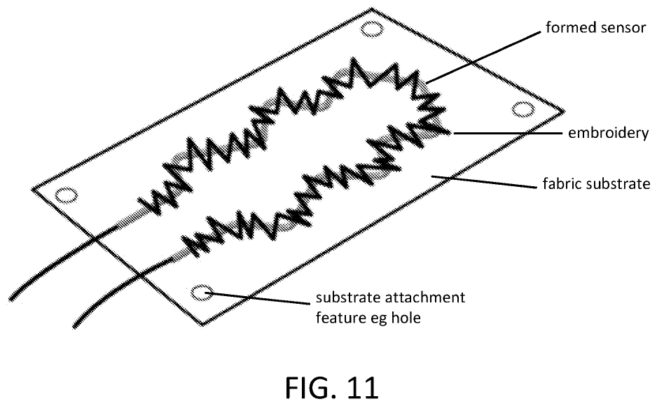

FIG. 11 shows an exemplary garment-sensor system, in accordance with some embodiments of the present disclosure. The garment-sensor system includes a fabric substrate and an optical fiber (e.g., optical fiber 106a, 106b in FIGS. 1-4). The optical fiber may be a freestanding optical fiber, such as discussed in relation to FIGS. 7A-7D and 9A-9D, form a sensor that is adhered to the fabric substrate with elastomeric adhesive and/or embroidery. The fabric substrate has suitable material properties, such as limited stretch in undesired directions, a desired color, a durability that withstands stretching, etc., that a specific material, such as a garment material, may not possess. The garment-sensor system is a modular object that can be applied to and removed from a material, such as through the substrate attachment features (e.g., holes formed in the fabric substrate). In some embodiments, the garment-sensor system is manufactured before the material, which may be a garment. In some embodiments, a non-fabric material may be used instead of the fabric substrate.

In some embodiments, the garment-sensor system is part of or coupled to a material. The garment-sensor system, including the optical fiber, is pulled in a stretch direction. As the optical fiber deforms and bend angles of the optical fiber increase, control circuitry (not shown) detects a change (e.g., decrease) in optical attenuation. The change in optical attenuation may correlate linearly with the deformation or displacement in the stretch direction. Thus, the garment-sensor system may be used to determine strain of the optical fiber, or more specifically, to the material or an object to which the material is coupled.

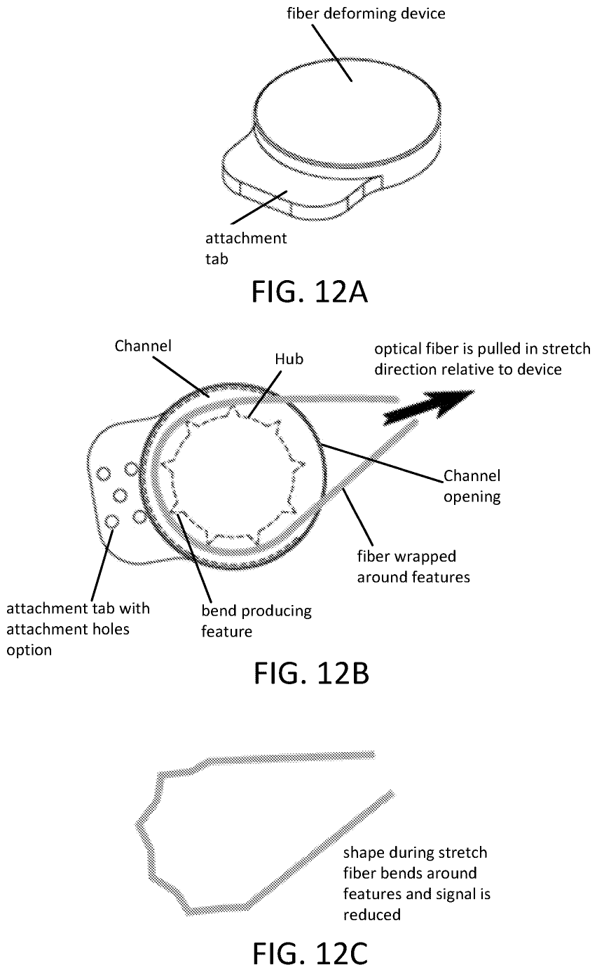

FIGS. 12A-12C show a fiber deforming device for shaping an optical fiber (e.g., optical fiber 106a, 106b in FIGS. 1-4), in accordance with some embodiments of the present disclosure.

Referring to FIG. 12A, the fiber deforming device includes an attachment tab to attach to a material or object. In some embodiments, the fiber deforming device is made from a flexible rubber or plastic that can be adhered to a fabric garment like similar button-like ornaments. Referring to FIG. 12B, the attachment tab may form attachment holes that are used to attach the fiber deforming device to the material. In some embodiments, the fiber deforming device is attached to one of a stretchable portion of the material (e.g., stretchable material 102 in FIGS. 1-4 or material in FIGS. 5A and 5B) or a non-stretchable portion of the material (e.g., non-stretchable portion 104 in FIGS. 1-4). The fiber deforming device comprises bend producing features. In the embodiment depicted in FIG. 12B, the bend producing features comprise a “star” hub or a gear-like hub having pointed teeth (shown in dashed line). The fiber deforming device has a lip that forms a channel (shown in dashed line) between the “star” hub, the lip, and the material. The fiber forming device forms a channel opening to allow access to the channel. The optical fiber is routed through the channel opening, disposed in the channel, and has a rounded shape when unstretched. Ends or end portions of the optical fiber may be pulled away from the fiber deforming device in a stretch direction.

Referring to FIG. 12C, the optical fiber may conform to the shape of the “star” hub when pulled in the stretch direction, and bend angles may form between sections of the optical fiber. When the optical fiber conforms to the hub shape, the optical signal is modulated as the bend angles are formed (e.g., from what is shown in FIG. 12B to FIG. 12C). In this case, the measured optical intensity through the optical fiber decreases. Thus, the fiber deforming device reduces the optical intensity (and increases optical attenuation) as the optical fiber is stretched. For example, the optical fiber is wrapped around the “star” hub when pulled and the features of the “star” hub produce distinct bends in the optical fiber having bend angles that decrease as the optical fiber is pulled.

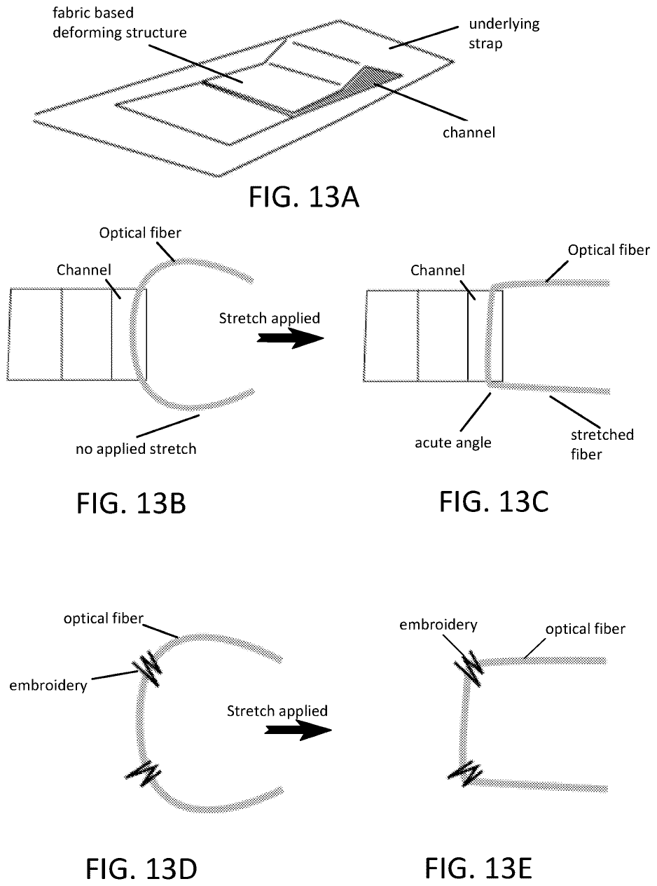

FIGS. 13A-13E show a fiber deforming structure for shaping an optical fiber (e.g., optical fiber 106a, 106b in FIGS. 1-4), in accordance with some embodiments of the present disclosure. The fiber deforming structure may be made from a soft material, such as a fabric depicted in FIGS. 13A-13C, or embroidered features as depicted in FIGS. 13D and 13E.

Referring to FIG. 13A, the fabric based fiber deforming structure is made from a material, such as a fabric, and forms a channel or loop in the material. The channel may be formed by folding the fabric on top of itself and attaching it to itself. The fabric based deforming structure may be attached to, or part of, an underlying strap. In some embodiments, the channel is formed by attaching the fabric to the underlying strap. The underlying strap is not depicted in FIGS. 13B and 13C. Referring to FIG. 13B, an optical fiber is disposed in or routed through the channel. The optical fiber forms an unstretched loop having a rounded shape that may be pulled in a stretch direction. Referring to FIG. 13C, as the optical fiber is stretched in the stretch direction, sharp corners are produced in the optical fiber at the outer edges of the channel and three linear sections of the optical fiber are formed that are about 90° from one another, such as 90°±5°, such as ±10° such as ±15°. The sharp corners in the optical fiber reduce the optical intensity. Thus, the fabric based fiber deforming device increases the optical attenuation as the optical fiber is stretched.

Referring to FIGS. 13D and 13C, a similar effect can be achieved by embroidering discrete locations along the unstretched loop of the optical fiber to a material. The embroidery forms an embroidery based fiber deforming device. In the embodiment depicted, two discrete locations of the optical fiber are embroidered to a material (not shown). In some embodiments, the material may be the underlying strap discussed in relation to FIG. 13A. As the optical fiber is stretched in a stretch direction, the optical fiber bends around these embroidered features and reduces the optical intensity. Thus, the embroidery based fiber deforming device increases the optical attenuation as the optical fiber is stretched.

The embodiments discussed above are intended to be illustrative and not limiting. One skilled in the art would appreciate that individual aspects of the apparatus and methods discussed herein may be omitted, modified, combined, and/or rearranged without departing from the scope of the disclosure. Only the claims that follow are meant to set bounds as to what the present disclosure includes.

What is claimed is:

1. A sensor system comprising:
a material having a stretchable region and a non-stretchable region; and
an optical fiber attached to the material, wherein:
fiber sections of the optical fiber are bonded together at a bond site on the optical fiber; and
the optical fiber is attached to the material such that the bond site is located in the non-stretchable region of the material.

2. The sensor system of claim 1, wherein a gradient between the stretchable region and the non-stretchable region of the material increases from a strain of around 0%-10% within the non-stretchable region and to a strain of up to 500% in the stretchable region.

3. The sensor system of claim 2, wherein the gradient increases in gradations of about 1%-10%.

4. The sensor system of any one of claims 2-3, wherein the gradient is based at least in part on stitch density.

5. The sensor system of any one of claims 1-4, wherein:
the fiber sections of the optical fiber are further bonded together at additional bond sites on the optical fiber; and
the optical fiber is attached to the material such that the additional bond sites on the optical fiber are located in the non-stretchable region of the material.

6. The sensor system of any one of claims 1-5, wherein the optical fiber is enclosed by a protective jacket.

7. The sensor system of any one of claims 1-6, wherein the optical fiber is configured to measure strain.

8. The sensor system of any one of claims 1-7, wherein the non-stretchable region is capable of 20% strain or less.

9. The sensor system of claim 8, wherein the non-stretchable region is capable of 15% strain or less.

10. The sensor system of any one of claims 1-9, wherein the non-stretchable region comprises a dense stitching pattern.

11. The sensor system of claim 10, wherein the dense stitching pattern comprises thread spacing of no more than 2 mm.

12. The sensor system of any one of claims 10-11, wherein the thread spacing of the dense stitching pattern is uniform.

13. The sensor system of any one of claims 10-11, wherein the thread spacing of the dense stitching pattern is non-uniform.

14. The sensor system of any one of claims 10-13, wherein the dense stitching pattern comprises an average thread spacing of no more than 2 mm.

15. The sensor system of any one of claims 10-13, wherein the dense stitching pattern comprises an average thread spacing of no more than 1 mm.

16. The sensor system of any one of claims 1-13, wherein the dense stitching pattern comprises an average thread spacing of no more than 0.5 mm.

17. The sensor system of any one of claims 1-16, wherein a first length of the optical fiber in the non-stretchable region on a first side of the bond site is at least 15 mm.

18. The sensor system of claim 17, wherein a second length of the optical fiber in the non-stretchable region on a second side of the bond site is at least 15 mm.

19. The sensor system of any one of claims 17-18, wherein the optical fiber is substantially straight along the first length.

20. The sensor system of any one of claims 17-19, wherein the optical fiber is substantially straight along the second length.

21. The sensor system of any one of claims 17-20, wherein a direction of the optical fiber along the first length is substantially perpendicular to a stretch direction of the material.

22. The sensor system of any one of claims 17-20, wherein a direction of the optical fiber along the first length is off-parallel to a stretch direction of the material.

23. The sensor system of claim 22, wherein an angle between the direction of the optical fiber along the first length and the stretch direction of the material is less than 45°.

24. The sensor system of claim 22, wherein an angle between the direction of the optical fiber along the first length and the stretch direction of the material is less than 20°.

25. The sensor system of any one of claims 1-24, wherein the optical fiber is attached to the material using stitching or using a polymer casing.

26. A sensor comprising:
a material capable of stretching along a stretch direction; and
an optical fiber attached to the material, wherein:
the optical fiber comprises linear sections along its length;
the linear sections are each angled relative to the stretch direction; and
when the material is stretched along the stretch direction,
the angles of the linear sections relative to the stretch direction decrease; and
optical attenuation of the optical fiber decreases.

27. The sensor of claim 26, wherein:
the linear sections are separated by bends having bend angles; and
when the material is stretched along the stretch direction,
the bend angles increase; and
the optical attenuation associated with each of the bends decreases.

28. The sensor of any one of claims 26-27, wherein linear sections of a first set of the linear sections are separated by alternating bends.

29. The sensor of claim 28, wherein linear sections of a second set of the linear sections are separated by alternating bends.

30. The sensor of claim 29, wherein the first set of the linear sections forms a first side of a loop and the second set of the linear sections forms a second side of the loop.

31. The sensor of claim 30, wherein the first set of the linear sections and the second set of linear sections are separated by an inner facing bend.

32. The sensor of claim 30, wherein the first set of linear sections and the second set of linear sections are connected via two linear sections separated by an outer facing bend.

33. The sensor of claim 30, wherein:
the first set of the linear sections comprises at least four linear sections; and
the second set of the linear sections comprises at least four linear sections.

34. The sensor of any one of claims 26-33, wherein when the material is unstretched, the linear sections are angled greater than 45° relative to the stretch direction.

35. The sensor of any one of claims 26-34, wherein the optical fiber comprises:
a first end configured to be coupled to a light emitter; and
a second end configured to be coupled to a light detector.

36. The sensor of any one of claims 26-35, wherein the optical fiber comprises at least three segments bonded together comprising a first end segment, a second end segment, and one or more middle segments.

37. The sensor of claim 36, wherein the one or more middle segments comprises the linear sections.

38. The sensor of any one of claims 36-37, wherein the one or more middle segments comprises a material having greater stretchability than material used in the first and second end segments.

39. The sensor of any one of claims 36-38, wherein the one or more middle segments comprises a material having less optical transmissibility than material used in the first and second end segments.

40. The sensor of any of claims 26-39, wherein the linear sections comprise at least nine linear sections.

41. The sensor of any of claims 26-40, wherein the optical fiber comprising the linear sections along its length is a formed optical fiber that is made by thermoforming the optical fiber.

42. A sensor comprising:
a material capable of stretching along a stretch direction; and
an optical fiber attached to the material, wherein:
the optical fiber comprises a plurality of alternating bends along its length;
the alternating bends comprise bend angles; and
when the material is stretched along the stretch direction,
the bend angles of the alternating bends increase; and
optical attenuation of the optical fiber decreases.

43. The sensor of claim 42, wherein when the material is unstretched, the bend angles are less than 50°.

44. The sensor of claim 42, wherein when the material is unstretched, the bend angles are less than 40°.

45. The sensor of any one of claims 42-44, wherein the optical fiber comprises a linear section between each adjacent pair of alternating bends.

46. The sensor of any one of claims 42-44, wherein a first set of at least 3 alternating bends form a first side of a loop and a second set of at least 3 alternating bends form a second side of the loop.

47. The sensor of any of claims 42-46, wherein the optical fiber comprising the plurality of alternating bends along its length is a formed optical fiber that is made by thermoforming the optical fiber.
